(19) United States
(12) Patent Application Publication
(10) Pub. No.: US 2023/0178210 A1
(43) Pub. Date: Jun. 8, 2023

(54) Acupuncture and Moxibustion Treatment Support Apparatus, Acupuncture and Moxibustion Treatment Support Method, Acupuncture and MoxibustionTreatment Support Program, and Acupuncture and Moxibustion Treatment Support System

(71) Applicant: Seirin Corporation, Shizuoka (JP)

(72) Inventors: Ryoichi Nakano, Shimizu (JP); Masahiro Kikuchi, Shimizu (JP)

(73) Assignee: Seirin Corporation, Shizuoka (JP)

(21) Appl. No.: 17/912,772

(22) PCT Filed: Aug. 18, 2021
(86) PCT No.: PCT/JP2021/030177

§ 371 Date: Sep. 19, 2022

(30) Foreign Application Priority Data
Aug. 28, 2020 (JP) 2020-144579

(51) Int. Cl. G16H 20/40; G16H 10/20; G16H 30/20

(52) U.S. Cl. CPC G16H 20/40; G16H 10/20; G16H 30/20

(57) ABSTRACT

A pre-treatment indicator corresponding to a symptom level of a patient before treatment and a post-treatment indicator corresponding to a symptom level of the patient after treatment are obtained and stored in a storage unit in association with information indicating a treatment technique used in acupuncture and moxibustion treatment, the pre-treatment indicator and the post-treatment indicator being entered based on selection by the patient from indicators representing symptom levels of a symptom treated by the acupuncture and moxibustion treatment, the patient receiving the acupuncture and moxibustion treatment. When the information indicating the treatment technique is specified, the treatment technique, the pre-treatment indicator, and the post-treatment indicator are read from the storage unit and are output.

### TECHNICAL FIELD

The present invention relates to an acupuncture and moxibustion treatment support apparatus, an acupuncture and moxibustion treatment support method, an acupuncture and moxibustion treatment support program, and an acupuncture and moxibustion treatment support system for supporting acupuncture and moxibustion treatment.

### BACKGROUND ART

In acupuncture and moxibustion treatment, an acupuncturist, who is a practitioner of acupuncture and moxibustion, identifies a symptom based on a primary complaint of a patient, determines a treatment method corresponding to the cause of the symptom, and provides appropriate treatment to alleviate the symptom of the patient. A technology for supporting the acupuncture and moxibustion treatment has been proposed for making it possible to accurately identify the symptom of a patient. It has been proposed to have an information processing apparatus cause a display unit to display a human figure schematically illustrating a human body with site marks representing sites of the human body in the human figure, and when a site corresponding to a disorder of the patient is selected from the sites, cause the display unit to display a list of disorder patterns related to the selected site.

### REFERENCE DOCUMENT LIST

### Patent Document

Patent Document 1: JP 2019-079515 A

### SUMMARY OF THE INVENTION

### Problem to be Solved by the Invention

In acupuncture and moxibustion treatment, after identifying the symptom of a patient, an acupuncturist determines a treatment method suitable for the symptom. When determining a treatment method, the acupuncturist selects an appropriate procedure and also selects a treatment site at which the procedure is to be performed among, for example, a large number of acupuncture points on the human body. Because there are diverse and complex combinations of procedures and treatment sites, highly specialized knowledge and a great deal of experience are necessary to determine a treatment method suitable for each patient.

On the other hand, however, it is often difficult to determine, from appearance, the level of a symptom to be treated by acupuncture and moxibustion. Accordingly, it is difficult for an acupuncturist to perceive how the symptom of the patient has been improved by treatment and to determine the effect of the treatment. Also, for this reason, it is difficult to determine the appropriateness of the treatment.

In view of the above problems, an object according to an aspect of the present invention is to enable an acupuncturist to accurately determine the level of a symptom of a patient in acupuncture and moxibustion treatment and thereby improve treatment accuracy.

### Means for Solving the Problem

According to an aspect of the present invention, a pre-treatment indicator corresponding to a symptom level of a patient before treatment and a post-treatment indicator corresponding to a symptom level of the patient after treatment are obtained and stored in a storage unit in association with information indicating a treatment technique used in acupuncture and moxibustion treatment, the pre-treatment indicator and the post-treatment indicator being entered based on selection by the patient from indicators representing symptom levels of a symptom treated by the acupuncture and moxibustion treatment, the patient receiving the acupuncture and moxibustion treatment. When the information indicating the treatment technique is specified, the treatment technique, the pre-treatment indicator, and the post-treatment indicator are read from the storage unit and are output.

### Effects of the Invention

One aspect of the present invention enables an acupuncturist to accurately determine the level of a symptom of a patient in acupuncture and moxibustion treatment and thereby makes it possible to improve treatment accuracy.

### BRIEF DESCRIPTION OF THE DRAWINGS

FIG. 1 is a block diagram illustrating an example of the overall configuration of an acupuncture and moxibustion treatment support system;

FIG. 2 is a sequence chart illustrating an example of an entire process performed by an acupuncture and moxibustion clinic terminal and a patient terminal in collaboration with each other;

FIG. 3 is a drawing illustrating an example of a process performed by an acupuncture and moxibustion clinic terminal;

FIG. 4 is a drawing illustrating an example of a patient list screen;

FIG. 5 is a drawing illustrating an example of a patient details screen;

FIG. 6 is a drawing illustrating an example of a preliminary examination sheet setting screen;

FIG. 7 is a drawing illustrating an example of a symptom setting screen;

FIG. 8 is a drawing illustrating examples of indicators representing symptom levels on a scale;

FIG. 9 is a drawing illustrating an example of an electronic medical record setting screen;

FIG. 10 is a drawing illustrating an example of a treatment technique setting screen;

FIG. 11 is a drawing illustrating an example of a patient support setting screen;

FIG. 12 is a drawing illustrating an example of an electronic medical record reference screen;

FIG. 13 is a drawing illustrating an example of a follow-up information screen;

FIG. 14 is a drawing illustrating an example of a statistical information screen;

FIG. 15 is a drawing illustrating an example of a process performed by a patient terminal;

FIG. 16 is a drawing illustrating an example of a patient basic screen;

FIG. 17 is a drawing illustrating an example of a question-and-answer screen;

FIG. 18 is a drawing illustrating an example of a process performed by a management server;

FIG. 19 is a drawing illustrating an example of an acupuncture and moxibustion clinic evaluation screen; and

FIG. 20 is a drawing illustrating an example of a hardware configuration of an information processing apparatus.

### MODES FOR CARRYING OUT THE INVENTION

### 1. Overall Configuration of System of Present Embodiment

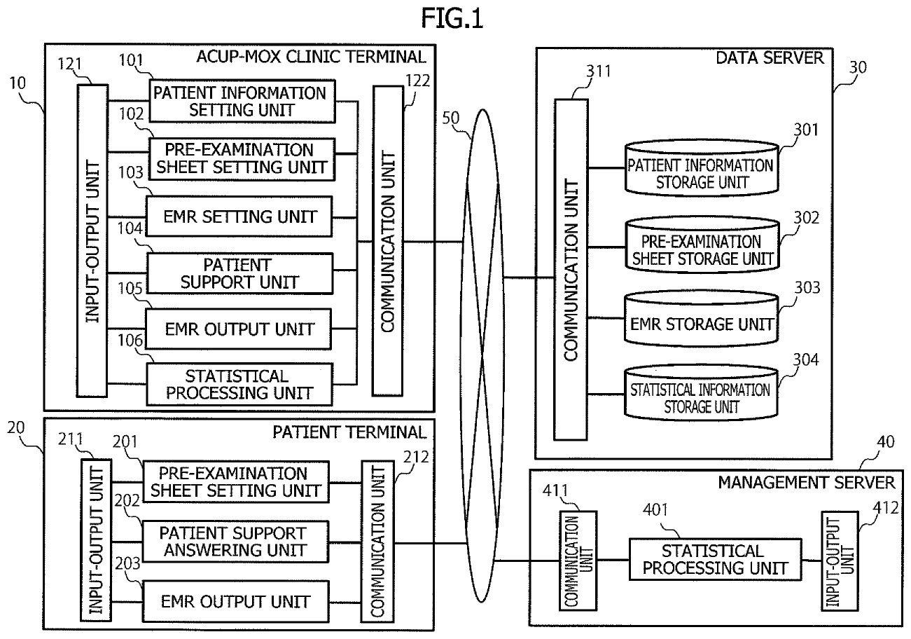

FIG. 1 illustrates an example of an overall configuration of an acupuncture and moxibustion (Acup-Mox) treatment support system according to the present embodiment. The acupuncture and moxibustion treatment support system includes an acupuncture and moxibustion clinic terminal 10, a patient terminal 20, a data server 30, and a management server 40. Each of the acupuncture and moxibustion clinic terminal 10, the patient terminal 20, the data server 30, and the management server 40 is an information processing apparatus and may be implemented by, for example, a desktop computer, a mobile computer, a tablet computer, a mobile device, or any other type of device. In the present embodiment, as an example, it is assumed that the acupuncture and moxibustion clinic terminal 10 is a tablet computer and the patient terminal 20 is a smartphone. The acupuncture and moxibustion clinic terminal 10, the patient terminal 20, the data server 30, and the management server 40 are interconnected via a network 50. The network 50 may include, for example, one or more of a local area network (LAN), a wide area network (WAN), the Internet, and the like. Although only one acupuncture and moxibustion clinic terminal 10 and one patient terminal 20 are illustrated in FIG. 1, there may be multiple acupuncture and moxibustion clinic terminals 10 and multiple patient terminals 20.

The acupuncture and moxibustion clinic terminal 10 is an information processing apparatus used at an acupuncture and moxibustion clinic that provides acupuncture and moxibustion treatment. It is assumed that the acupuncture and moxibustion clinic terminal 10 is used mainly by an acupuncturist (a practitioner of acupuncture and moxibustion), and the present embodiment is described based on an assumption that the user of the acupuncture and moxibustion clinic terminal 10 is basically an acupuncturist. However, the present invention is not limited to this example, and the acupuncture and moxibustion clinic terminal 10 may also be used by a patient or other employees as necessary. The acupuncture and moxibustion clinic terminal 10 includes a patient information setting unit 101, a preliminary examination sheet setting unit 102, an electronic medical record (EMR) setting unit 103, a patient support unit 104, an electronic medical record output unit 105, and a statistical processing unit 106, the functions of which are implemented by executing an acupuncture and moxibustion treatment support program on the acupuncture and moxibustion clinic terminal 10. The acupuncture and moxibustion clinic terminal 10 also includes an input-output unit 121 and a communication unit 122.

The patient information setting unit 101 sets, in a patient information storage unit 301 of the data server 30, basic information about a patient that is entered via a user interface and includes, for example, the name, the sex, the age, the occupation, and the contact information of the patient. Normally, the patient information is set when a treatment appointment is made for a new patient at an acupuncture and moxibustion clinic.

The preliminary examination sheet setting unit 102 generates, based on information indicating a symptom of a patient before treatment and entered via a user interface, a preliminary examination sheet that includes the information and is unique to the patient, and stores the preliminary examination sheet in a preliminary examination sheet storage unit 302 of the data server 30. The information indicating the symptom in the preliminary examination sheet includes the onset date of the symptom and an indicator (pre-treatment indicator) that corresponds to the level of the symptom of the patient before treatment and is entered based on a selection by the patient from indicators representing symptom levels on a scale.

The electronic medical record setting unit 103 generates an electronic medical record of a patient when acupuncture and moxibustion treatment is provided for the patient, and stores the electronic medical record in the electronic medical record storage unit 303 of the data server 30. The electronic medical record includes, for example, patient information of the patient in association with a symptom of the patient and information indicating a treatment technique (including a treatment site for the symptom, a procedure performed at the treatment site, and acupuncture and moxibustion information corresponding to the procedure) that are entered via a user interface. The symptom of the patient includes information such as an onset date of the symptom, and a pre-treatment indicator and an indicator (post-treatment indicator) corresponding to a level of the symptom after treatment that are entered based on selections by the patient from indicators representing symptom levels on a scale. The indicator corresponding to the symptom of the patient indicates one of “indicators representing symptom levels on a scale” that is closest to the symptom of the patient. Also, the electronic medical record may include a pre-treatment image indicating the symptom of the patient before treatment and a post-treatment image indicating the symptom of the patient after treatment (for example, images showing an affected area of the patient before and after treatment). Furthermore, the electronic medical record may include information indicating a post-treatment indicator obtained after treatment by the patient support unit 104 described later from the patient terminal 20 and an input timing of the post-treatment indicator. Here, a symptom in the present embodiment indicates information that also includes a site with, for example, pain. The electronic medical record setting unit 103 is an example of a treatment information setting unit.

After acupuncture and moxibustion treatment is performed, the patient support unit 104 sends, to the patient terminal 20, a question about the level of a symptom after the treatment. The patient can answer the question by selecting an indicator corresponding to the current symptom of the patient from indicators representing symptom levels on a scale and by entering the selected indicator as a post-treatment indicator. The timing (e.g., a transmission period and a transmission interval (frequency)) of sending the question and the scale of levels of the symptom (the number of levels) may be set when entering an electronic medical record. When receiving, from the patient terminal 20, information indicating the post-treatment indicator of the patient and its input timing as an answer to the question, the patient support unit 104 stores the information in the electronic medical record storage unit 303 in association with the electronic medical record.

The electronic medical record output unit 105 reads an electronic medical record stored in the electronic medical record storage unit 303 of the data server 30 and outputs the electronic medical record to an output unit of the acupuncture and moxibustion clinic terminal 10. The output unit is, for example, a display or a printer. The electronic medical record output unit 105 is an example of a treatment information output unit.

The statistical processing unit 106 performs statistical processing based on information such as treatment techniques included in electronic medical records, pre-treatment indicators, and/or post-treatment indicators stored in a storage unit of the data server 30, and outputs a processing result via an output unit. Also, the statistical processing unit 106 stores the processing result in a statistical information storage unit 304 as necessary.

The input-output unit 121 is implemented by, for example, various drivers that function as interfaces for an input device and an output device included in the acupuncture and moxibustion clinic terminal 10. The communication unit 122 is a communication module that functions as an interface for enabling communications between the acupuncture and moxibustion clinic terminal 10 and the network 50.

The patient terminal 20 is used by a patient who receives acupuncture and moxibustion treatment. The patient terminal 20 includes a preliminary examination sheet setting unit 201, a patient support answering unit 202, and an electronic medical record output unit 203 the functions of which are implemented by executing the acupuncture and moxibustion treatment support program on the patient terminal 20. Also, the patient terminal 20 includes an input-output unit 211 and a communication unit 212.

Similarly to the preliminary examination sheet setting unit 102 of the acupuncture and moxibustion clinic terminal 10, the preliminary examination sheet setting unit 201 generates, based on information that indicates, for example, a symptom of a patient before treatment and is input via a user interface, a preliminary examination sheet that includes the information and is unique to the patient, and stores the preliminary examination sheet in the preliminary examination sheet storage unit 302 of the data server 30.

Thus, preliminary examination sheets in the preliminary examination sheet storage unit 302 can be set from both of the acupuncture and moxibustion clinic terminal 10 and the patient terminal 20. For example, a patient can set a preliminary examination sheet from the patient terminal 20 before receiving treatment and can also set a preliminary examination sheet using the acupuncture and moxibustion clinic terminal 10 after arriving at an acupuncture and moxibustion clinic to receive treatment. Also, for example, a preliminary examination sheet may be set by an acupuncturist using the acupuncture and moxibustion clinic terminal 10 based on an interview with a patient.

The patient support answering unit 202 displays, on a display of a touch panel, a user interface for answering a question related to a symptom after treatment and sent from the patient support unit 104 of the acupuncture and moxibustion clinic terminal 10. The user interface is configured such that the question can be answered using an indicator representing a level of the symptom. A described above, the patient can answer the question by selecting an indicator corresponding to the current symptom of the patient from indicators representing symptom levels on a scale and by entering the selected indicator as a post-treatment indicator. The patient support answering unit 202 performs an answering process based on the post-treatment indicator entered by the patient.

The electronic medical record output unit 203 reads an electronic medical record stored in the electronic medical record storage unit 303 of the data server 30 and outputs the electronic medical record to an output unit. The output unit is, for example, a display or a printer.

The input-output unit 211 is implemented by, for example, various drivers that function as interfaces with an input device and an output device provided in the patient terminal 20.

The communication unit 212 is a communication module that functions as an interface for enabling communication between the patient terminal 20 and the network 50.

The data server 30 includes the patient information storage unit 301, the preliminary examination sheet storage unit 302, the electronic medical record storage unit 303, and the statistical information storage unit 304 that are implemented by a storage device (storage unit) provided in the data server 30. The data server 30 also includes a communication unit 311.

In the patient information storage unit 301, patient information, which is basic information about each patient, is stored in advance. The patient information includes, for example, name, sex, age, occupation, and contact information of the patient.

The preliminary examination sheet storage unit 302 stores information on preliminary examination sheets of individual patients. For example, a preliminary examination sheet includes patient information, a symptom of a patient before treatment, medical history, and lifestyle. The symptom of the patient includes information indicating the onset date of the symptom and a pre-treatment indicator.

The electronic medical record storage unit 303 stores information on electronic medical records of individual patients. An electronic medical record includes information such as patient information, a treatment date, an attending acupuncturist, a symptom of a patient, and a treatment technique. As described above, the symptom of a patient includes information indicating the onset date of the symptom, and a pre-treatment indicator and a post-treatment indicator of the symptom. The post-treatment indicator may be obtained from the patient terminal 20. The electronic medical record also includes an access control flag for controlling the access right from the patient terminal 20.

The statistical information storage unit 304 stores processing results (statistical information) of statistical processing performed by the statistical processing unit 106.

When the present system includes multiple acupuncture and moxibustion clinic terminals 10 used in multiple acupuncture and moxibustion clinics, for example, an identifier may be assigned to each set of data stored in the patient information storage unit 301, the preliminary examination sheet storage unit 302, the electronic medical record storage unit 303, and the statistical information storage unit 304 to indicate an acupuncture and moxibustion clinic to which the data belongs. In this case, it is assumed that each acupuncture and moxibustion clinic terminal 10 can access only data belonging to an acupuncture and moxibustion clinic where the acupuncture and moxibustion clinic terminal 10 is used. Similarly, it is assumed that a patient terminal 20 used by a patient of an acupuncture and moxibustion clinic can access only data belonging to the acupuncture and moxibustion clinic where the patient using the patient terminal 20 receives treatment.

The communication unit 311 is a communication module that functions as an interface for enabling communications between the data server 30 and the network 50.

The management server 40 can access the storage device of the data server 30. The management server 40 is mainly used by a system administrator of the acupuncture and moxibustion treatment support system. Here, a system administrator not only indicates a person who maintains and manages the operation of the system, but may also indicate any person who is allowed to use information stored in the storage device of the data server 30. The management server 40 includes the statistical processing unit 401 the function of which is implemented by executing a part of the acupuncture and moxibustion treatment support program on the management server 40. The management server 40 also includes a communication unit 411 and an input-output unit 412.

The statistical processing unit 401 accesses the data server 30 via the communication unit 411, performs, similarly to the statistical processing unit 106 of the acupuncture and moxibustion clinic terminal 10, statistical processing based on information stored in the storage device of the data server 30 such as information indicating treatment techniques included in electronic medical records, pre-treatment indicators, and post-treatment indicators, and outputs a processing result via an output unit. Here, different from the statistical processing unit 106 of the acupuncture and moxibustion clinic terminal 10, the statistical processing unit 401 can perform statistical processing on data of all acupuncture and moxibustion clinics. Then, the statistical processing unit 401 outputs a processing result of the statistical processing via the output unit. The output unit is, for example, a display or a printer. The statistical processing unit 401 can also store the processing result in the statistical information storage unit 304 as necessary.

The communication unit 411 is a communication module that functions as an interface for enabling communications between the management server 40 and the network 50. The input-output unit 412 is implemented by, for example, various drivers that function as interfaces with an input device and an output device provided in the management server 40.

### 2. Processes Performed by Program

Next, processes performed, respectively, by the acupuncture and moxibustion clinic terminal 10 and the patient terminal 20 are described.

### 2.1. Overview of Process in Acupuncture and Moxibustion Clinic Terminal and Patient Terminal

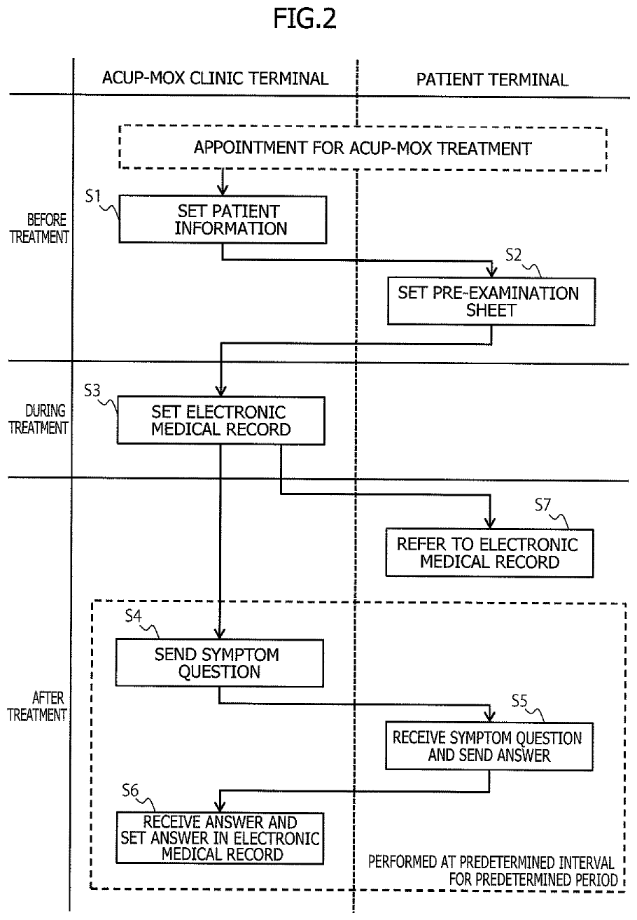

FIG. 2 is a sequence chart illustrating an example of an entire process performed by the acupuncture and moxibustion clinic terminal 10 and the patient terminal 20 in collaboration with each other. In acupuncture and moxibustion treatment, a patient first makes a treatment appointment for acupuncture and moxibustion treatment with an acupuncture and moxibustion clinic. The treatment appointment may be made, for example, using the present system or by telephone. Although it is possible to configure the present system to include a function for managing treatment appointments, descriptions of such a function are omitted in this specification.

When the treatment appointment is made, an acupuncturist of the acupuncture and moxibustion clinic operates the acupuncture and moxibustion clinic terminal 10 to set patient information. Based on the patient information entered by the operation, the patient information setting unit 101 of the acupuncture and moxibustion clinic terminal 10 stores the patient information in the patient information storage unit 301 (S1 in FIG. 2). Here, when the treatment appointment of the patient is the second or subsequent time and the patient information has already been registered, setting of the patient information is not necessary. It is also possible to provide a function to enable the patient terminal 20 to set patient information.

When the patient information is set, the patient information setting unit 101 of the acupuncture and moxibustion clinic terminal 10 reports to the patient terminal 20 that entry of a preliminary examination sheet has become possible. In response, the patient operates the patient terminal 20 to set a preliminary examination sheet. Based on information entered by the patient and indicating a symptom of the patient before treatment, the preliminary examination sheet setting unit 201 of the patient terminal 20 generates a preliminary examination sheet that includes the information and unique to the patient, and stores the preliminary examination sheet in the preliminary examination sheet storage unit 302 (S2). When the preliminary examination sheet is set, the preliminary examination sheet storage unit 302 reports to the acupuncture and moxibustion clinic terminal 10 that the preliminary examination sheet has been set. As a result, it becomes possible to obtain and refer to the preliminary examination sheet set by the patient by using the acupuncture and moxibustion clinic terminal 10. Here, as described above, with the function of the preliminary examination sheet setting unit 102 of the acupuncture and moxibustion clinic terminal 10, the patient can also enter the preliminary examination sheet by using the acupuncture and moxibustion clinic terminal 10 after arriving at the acupuncture and moxibustion clinic for treatment, and also the acupuncturist can enter the preliminary examination sheet for the patient.

When the patient visits the acupuncture and moxibustion clinic on a treatment date to receive acupuncture and moxibustion treatment, the acupuncturist operates the acupuncture and moxibustion clinic terminal 10 to set an electronic medical record. When electronic medical record information is entered, the patient support unit 104 of the acupuncture and moxibustion clinic terminal 10 receives settings for the timing when a question about a symptom after treatment is sent to the patient and includes the settings in the electronic medical record information. Such settings related to a question may be set for each electronic medical record (for each case of treatment for each patient), or the same settings may be set in advance for all cases of treatment of all patients. Then, the electronic medical record setting unit 103 of the acupuncture and moxibustion clinic terminal 10 generates the electronic medical record including the electronic medical record information entered by the operation and the settings related to the question, and stores the electronic medical record in the electronic medical record storage unit 303 (S3).

Then, after the treatment, the patient support unit 104 of the acupuncture and moxibustion clinic terminal 10 sends the question about a symptom after treatment to the patient terminal 20 at the set timing (S4).

At the patient terminal 20, when the question is received, the patient selects an indicator corresponding to the current level of the symptom from indicators representing symptom levels on a scale and enters the selected indicator as a post-treatment indicator. The patient support answering unit 202 of the patient terminal 20 sends, to the acupuncture and moxibustion clinic terminal 10, the post-treatment indicator entered by the patient as an answer to the question together with information indicating the input timing of the post-treatment indicator (S5). When receiving the answer, the patient support unit 104 of the acupuncture and moxibustion clinic terminal 10 further stores information contained in the answer in the electronic medical record storage unit 303 (S6).

Steps S4 through S6 are repeated until the set transmission timing of the question is completed. For example, when a transmission period and a transmission interval are set as the transmission timing, steps S4 through S6 are repeated at the set transmission interval until the set transmission period ends.

In short, at steps S4 through S6 described above, the patient terminal 20 provides a post-treatment indicator to the acupuncture and moxibustion clinic terminal 10 together with information indicating the input timing of the post-treatment indicator in response to a question from the acupuncture and moxibustion clinic terminal 10, and the acupuncture and moxibustion clinic terminal 10 obtains the provided information. In steps S4 through S6, the method of answering the question by the patient support answering unit 202 is not limited to the example described above. As another example, the patient support answering unit 202 may answer the question by additionally storing the information indicating the post-treatment indicator and its input timing in the electronic medical record storage unit 303. Even with this method, it is possible to implement a process in which the patient terminal 20 provides a post-treatment indicator and information indicating the input timing of the post-treatment indicator to the acupuncture and moxibustion clinic terminal 10, and the acupuncture and moxibustion clinic terminal 10 obtains the provided information. In this case, as necessary, the patient terminal 20 may send a report indicating that the question has been answered, to the patient support unit 104 of the acupuncture and moxibustion clinic terminal 10.

Once the electronic medical record is stored in the electronic medical record storage unit 303, the electronic medical record output unit 203 of the patient terminal 20 can also read the electronic medical record (S7). This enables the patient to refer to the electronic medical record set by the acupuncturist by using the patient terminal 20 (S7).

### 2.2. Details of Process Performed by Acupuncture and Moxibustion Clinic Terminal

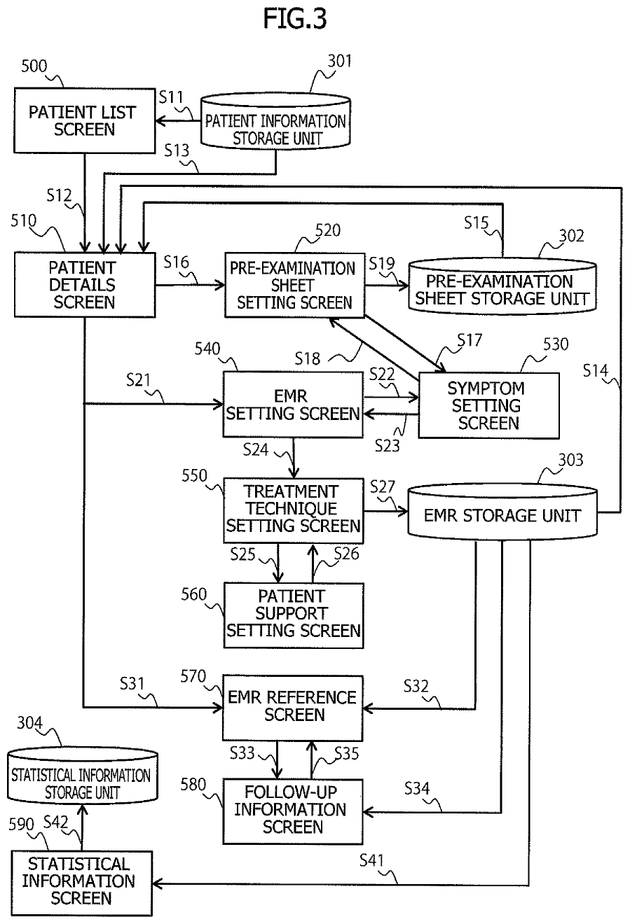

Next, processes performed by the patient information setting unit 101, the preliminary examination sheet setting unit 102, the electronic medical record setting unit 103, the patient support unit 104, the electronic medical record output unit 105, and the statistical processing unit 106 of the acupuncture and moxibustion clinic terminal 10 are described in detail with reference to examples of screens displayed in these processes. FIG. 3 is a drawing illustrating examples of processes performed by these units, along with screen transitions and access to storage units.

### 2.2.1. Patient Information Setting Process

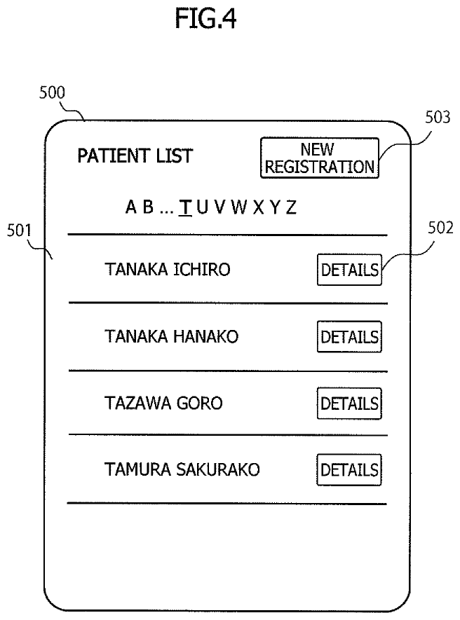

FIG. 4 illustrates an example of a patient list screen 500 displayed by the patient information setting unit 101. The patient list screen 500 displays a patient list 501 that is a list of patient names in patient information stored in the patient information storage unit 301 (S11 in FIG. 3). Each row of patient information in the patient list 501 displays a details button 502 for referring to the details of the corresponding patient information. Also, the patient list screen 500 displays a new registration button 503 for registering new patient information. In this specification, descriptions of the function of the patient information setting unit 101 for newly registering patient information are omitted.

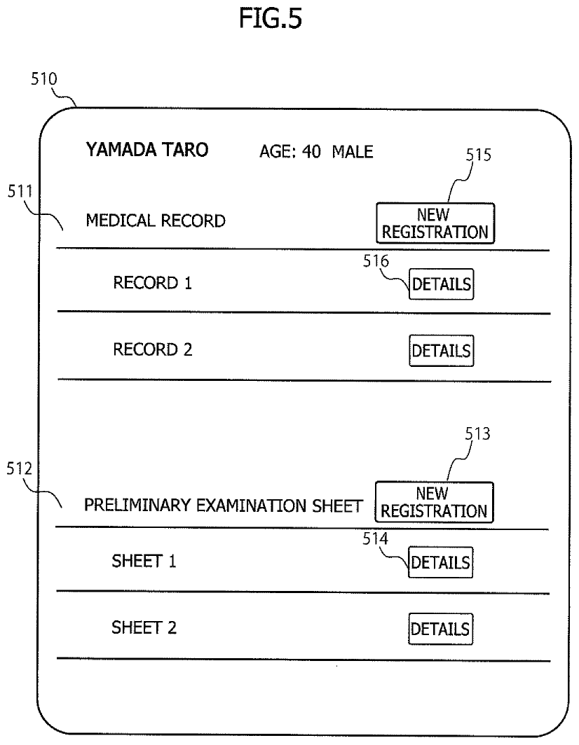

When the details button 502 in the patient list 501 is pressed by the acupuncturist, the patient information setting unit 101 displays a patient details screen 510 illustrated as an example in FIG. 5 (S12). Also, the patient information setting unit 101 reads patient information from the patient information storage unit 301 and displays the read patient information on the patient details screen 510 (S13). Although only name, age, and sex are displayed as an example of patient information in FIG. 5, more detailed information may be displayed. Also, on this screen, the electronic medical record setting unit 103 reads information on electronic medical records of the patient already stored in the electronic medical record storage unit 303 and displays the electronic medical records as an electronic medical record list 511 (S14). Furthermore, the preliminary examination sheet setting unit 102 reads information on preliminary examination sheets of the patient already stored in the preliminary examination sheet storage unit 302 and displays the preliminary examination sheets as a preliminary examination sheet list 512 (S15).

### 2.2.2. Preliminary Examination Sheet Setting Process

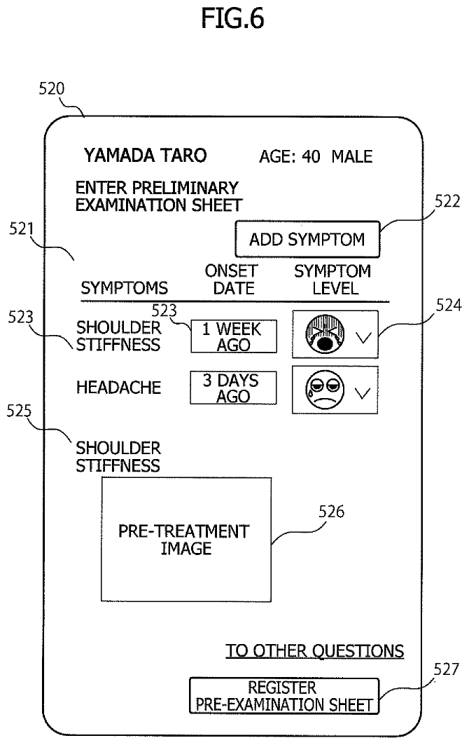

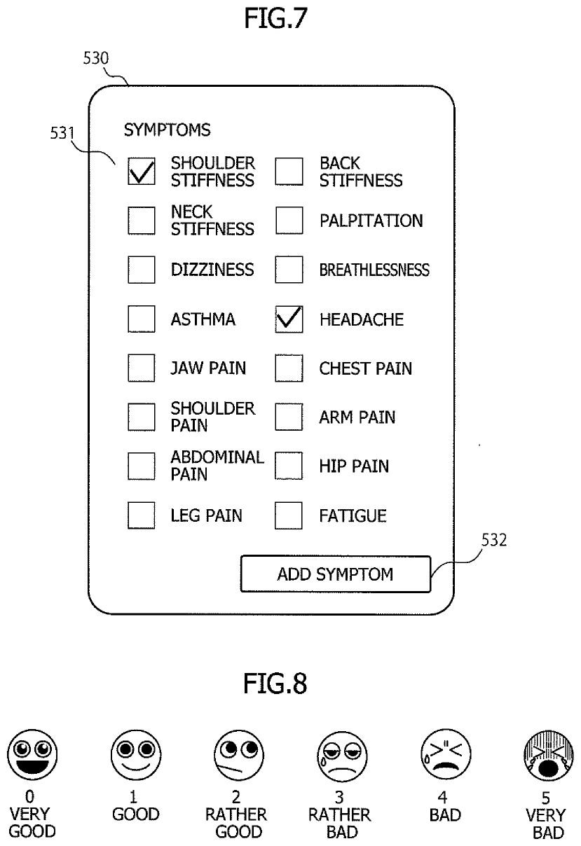

When the new registration button 513 for registering a preliminary examination sheet is pressed by the acupuncturist on the patient details screen 510, the preliminary examination sheet setting unit 102 displays a preliminary examination sheet setting screen 520 illustrated as an example in FIG. 6 (S16). For example, the preliminary examination sheet may be entered, before treatment for the patient is started, either by the patient visiting the acupuncture and moxibustion clinic or by the acupuncturist based on hearing from the patient. The preliminary examination sheet setting screen 520 displays, in addition to the patient information, an input field “symptoms 521” and a symptom addition button 522. When the symptom addition button 522 is pressed, the preliminary examination sheet setting unit 102 displays a symptom setting screen 530 illustrated as an example in FIG. 7 (S17). The symptom setting screen 530 displays a symptom list 531 of symptoms to be treated by acupuncture and moxibustion treatment together with check boxes. The acupuncturist or the patient selects one or more symptoms displayed in the symptom list 531 that correspond to symptoms of the patient before treatment, and presses a symptom addition button 532. Then, the preliminary examination sheet setting unit 102 returns to the preliminary examination sheet setting screen 520 and displays the selected symptoms in the symptom list 523 on the preliminary examination sheet setting screen 520 (S18). In the symptom list 523, the acupuncturist or the patient can enter the onset date of each symptom in an input field “onset date 523”. Also, in an input field “symptom level 524”, a pre-treatment indicator corresponding to the symptom before treatment can be selected from indicators representing symptom levels in a scale to enter a symptom level (in the descriptions below, the same applies to each case in which an indicator representing a symptom level is entered). As described above, the pre-treatment indicator in the symptom level 524 can be entered either by the acupuncturist by hearing about the level of the symptom from the patient or directly by the patient. In either case, the pre-treatment indicator is selected by the patient from indicators representing symptom levels on a scale.

FIG. 8 illustrates examples of indicators representing symptom levels on a scale. In the present embodiment, the scale includes six symptom levels: “0: Very good”, “1: Good”, “2: Rather good”, “3: Rather bad”, “4: Bad”, and “5: Very bad”. Also, as indicators representing the symptom levels, icons (FRS: Face Rating Scale) expressing the symptom levels with facial expressions are used. Here, the type of icons used as indicators representing symptom levels is not limited to FRS, and icons with any expressions and appearances may be used as long as they can visually indicate levels (intensities or degrees). The number of symptom levels in the scale may be set, in advance, as a uniform value in the program, or may be freely set by the acupuncturist on, for example, a setting screen.

Referring back to FIG. 6, the preliminary examination sheet setting screen 520 further displays an input field “image 525” and an image field 526 for setting a pre-treatment image for each symptom. When the image field 526 is tapped, a screen (not shown) that enables selection of an image is displayed, and the acupuncturist can add, for example, a photograph of an affected area of a patient. The added image is displayed in the image field 526. In the example of the preliminary examination sheet setting screen 520 of FIG. 6, although two symptoms including shoulder stiffness and headache are set, only an image input field for shoulder stiffness is displayed due to space limitations. Also, more detailed information (for example, medical history, lifestyle, etc.) can be entered on another screen by tapping a link “To Other Questions” on the preliminary examination sheet setting screen 520. However, descriptions of this function are omitted in this specification.

When a preliminary examination sheet registration button 527 on the preliminary examination sheet setting screen 520 is pressed by the acupuncturist, the preliminary examination sheet setting unit 102 obtains information that is entered on the preliminary examination sheet setting screen 520 and includes entries related to symptoms (including onset dates, pre-treatment indicators, and images), generates a preliminary examination sheet by associating the obtained information with the patient information, and stores the preliminary examination sheet in the preliminary examination sheet storage unit 302 (S19).

Here, on the patient details screen 510 in FIG. 5, when the acupuncturist presses the details button 514 in the row of each preliminary examination sheet, the preliminary examination sheet setting unit 102 reads detailed information of the preliminary examination sheet stored in the preliminary examination sheet storage unit 302, and displays the detailed information on another screen on which the acupuncturist can edit or delete the preliminary examination sheet as necessary. However, descriptions of this function are omitted in this specification.

### 2.2.3. Setting Process for Electronic Medical Record and Patient Support

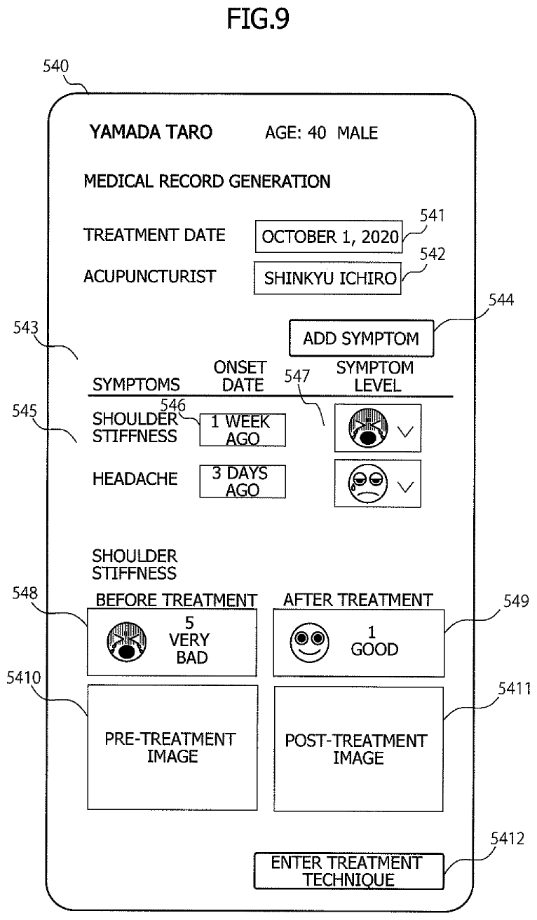

When a new registration button 515 for registering a new medical record is pressed by the user on the patient details screen 510 illustrated as an example in FIG. 5, the electronic medical record setting unit 103 displays an electronic medical record setting screen 540 illustrated as an example in FIG. 9 (S21 in FIG. 3). The electronic medical record setting screen 540 displays, in addition to patient information, an input field “treatment date 541” and an input field “attending acupuncturist 542”.

Also, the electronic medical record setting screen 540 displays an input field “symptoms 543” and a symptom addition button 544. When the symptom addition button 544 is pressed, as in the case of entering a preliminary examination sheet, the electronic medical record setting unit 103 displays the symptom setting screen 530 illustrated as an example in FIG. 7 (S22). The acupuncturist selects symptoms corresponding to the symptoms of the patient before treatment from symptoms displayed in the symptom list 531 on the symptom setting screen 530, and presses the symptom addition button 532. Then, the electronic medical record setting unit 103 returns to the electronic medical record setting screen 540 and displays the selected symptoms in the symptom list 545 on the electronic medical record setting screen 540 (S23). In the symptom list 545, the acupuncturist enters the onset date of each symptom in an input field “onset date 546”. Also, the acupuncturist can enter a pre-treatment indicator for each symptom in an input field “symptom level 547”. Here, when a preliminary examination sheet corresponding to the treatment to be recorded in the electronic medical record is stored in the preliminary examination sheet storage unit 302, information entered as symptoms in the corresponding preliminary examination sheet may be automatically applied to the symptom list 545 without change.

The electronic medical record setting screen 540 also displays input fields “pre-treatment indicator 548” and “post-treatment indicator 549” for each symptom. Here, because the pre-treatment indicator 548 corresponds to the entry in the symptom level 547, the entry may be automatically applied to the pre-treatment indicator 548 when the symptom level 547 is entered. For example, when treatment for a patient is finished, the acupuncturist can enter a post-treatment indicator in the post-treatment indicator 549 by hearing the level of each symptom of the patient after treatment (immediately after treatment). Alternatively, the patient may directly enter a post-treatment indicator in the input field “post-treatment indicator 549”. Also, instead of entering a post-treatment indicator in the post-treatment indicator 549 on the electronic medical record setting screen 540 of the acupuncture and moxibustion clinic terminal 10, information on a post-treatment indicator may be provided later by sending the post-treatment indicator corresponding to an entry in the post-treatment indicator 549 to the acupuncture and moxibustion clinic terminal 10 and requesting the acupuncture and moxibustion clinic terminal 10 to store the post-treatment indicator in a storage unit according to an operation of the patient terminal 20. In any case, the post-treatment indicator to be entered in the post-treatment indicator 549 is selected by the patient from multiple indicators representing symptom levels on a scale.

The electronic medical record setting screen 540 also displays an input field “image 549” and displays, for each symptom, a pre-treatment image field 5410 and a post-treatment image field 5411 for setting a pre-treatment image and a post-treatment image, respectively. Here, the pre-treatment image set in the preliminary examination sheet may be automatically applied to the pre-treatment image field 5410 without change. When each of the pre-treatment image field 5410 and the post-treatment image field 5411 is tapped, a screen (not shown) that enables selection of an image is displayed, and the acupuncturist can add a photograph of, for example, an affected area of the patient. The added image is displayed in the corresponding one of the pre-treatment image field 5410 and the post-treatment image field 5411. In the example of the electronic medical record setting screen 540 of FIG. 9, although two symptoms including shoulder stiffness and headache are set, only image input fields for shoulder stiffness are displayed due to space limitations.

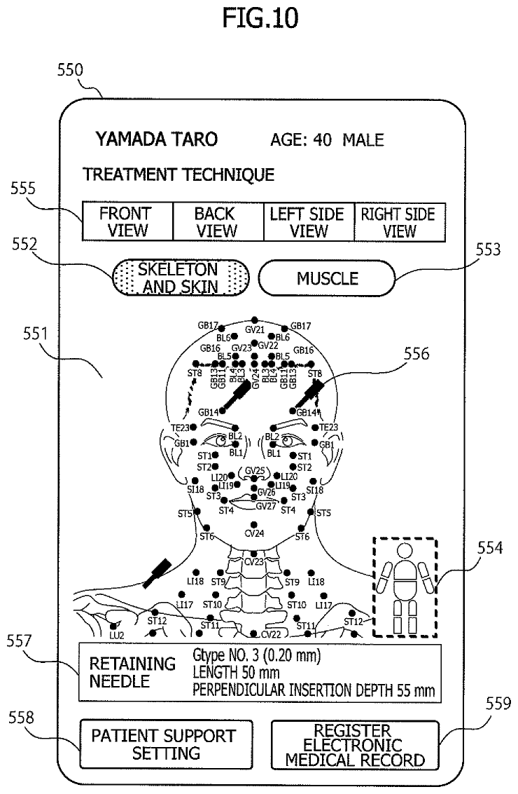

The electronic medical record setting screen 540 further displays a treatment technique input button 5412 for entering a treatment technique. When the treatment technique input button 5412 is pressed by the acupuncturist, the electronic medical record setting unit 103 displays a treatment technique setting screen 550 illustrated as an example in FIG. 10 (S24). The treatment technique setting screen 550 displays, in addition to patient information, a treatment input field 551 for entering a treatment technique. In the treatment input field 551, a skeleton and skin button 552, a muscle button 553, a human body mark 554, and display orientation switching buttons 555 are displayed. The display of the human figure can be changed by operating these buttons. Also, when the acupuncturist specifies a new treatment site on the human figure by an operation such as tapping, an icon 556 of “retaining needle” is displayed at the treatment site as a default value. Then, one of multiple sets of acupuncture and moxibustion information including a procedure in acupuncture and moxibustion treatment and details of an acupuncture and moxibustion product used in the procedure is displayed as default values in an acupuncture and moxibustion information field 557 in a lower part of the screen. Also, when an existing icon 556 is specified by an operation such as tapping, a procedure and acupuncture and moxibustion information set for the corresponding treatment site are displayed. When it is desired to set, in a treatment technique, a procedure and/or acupuncture and moxibustion information different from the default values or the set values, the acupuncturist can display an acupuncture and moxibustion information input screen (not shown) by tapping the acupuncture and moxibustion information field 557 and change, for example, the acupuncture and moxibustion information.

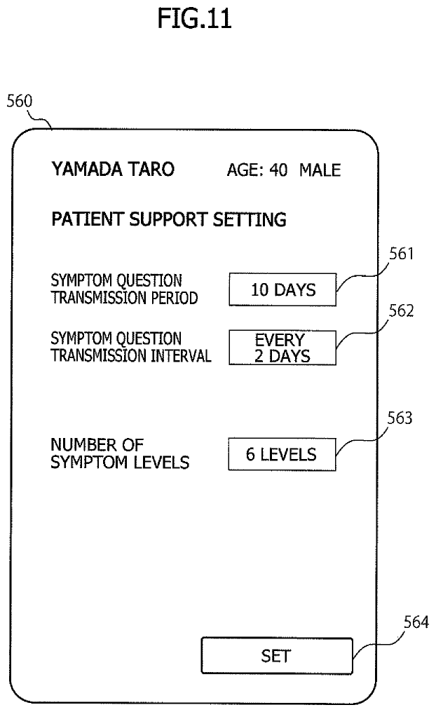

Here, the treatment technique setting screen 550 displays a patient support setting button 558 for setting a patient support function. When the patient support setting button 558 is pressed, the patient support unit 104 displays a patient support setting screen 560 illustrated as an example in FIG. 11 (S25). The patient support setting screen 560 displays input fields “symptom question transmission period 561” and “symptom question transmission interval 562” as examples of items for setting the timing of transmitting a question about a symptom after treatment. The acupuncturist can enter a transmission period and a transmission interval of a question for the patient by using these input fields. Also, the patient support setting screen 560 displays an input field “number of symptom levels 563” for setting the number of symptom levels in a scale. The number of symptom levels corresponds to the number of options from which the patient selects a post-treatment indicator as an answer to a question about a symptom. On a screen of the patient terminal 20 for answering the question, an indicator representing a symptom level can be selected from indicators, the number of which corresponds to the value entered in the input field “number of symptom levels 563”. In other words, the acupuncture and moxibustion clinic terminal 10 can receive, from the patient terminal 20, an answer including an indicator representing one of symptom levels in the scale. Then, when entry of all items is completed and a setting button 564 is pressed on the patient support setting screen 560, the patient support unit 104 stores the entries of the transmission period 561, the transmission interval 562, and the number of symptom levels 563, and returns to the treatment technique setting screen 550 (S26).

When the acupuncturist completes entry of all items on the treatment technique setting screen 550 and presses an electronic medical record registration button 559, the electronic medical record setting unit 103 obtains information that is entered on the electronic medical record setting screen 540, the treatment technique setting screen 550, and the patient support setting screen 560 and includes the treatment date, the attending acupuncturist, the symptoms (including the onset dates, the pre-treatment indicators, the post-treatment indicators, and the images), the treatment techniques (including the treatment sites, and the procedures and the acupuncture and moxibustion information for the respective treatment sites), and the patient support settings including the transmission period and the transmission interval of the question for the patient and the number of symptom levels, generates an electronic medical record by associating the obtained information with the patient information, and stores the electronic medical record in the electronic medical record storage unit 303 (S27).

Here, when the electronic medical record including the patient support settings is stored in the electronic medical record storage unit 303, as described above with reference to FIG. 2, the patient support unit 104 sends a question related to symptoms after treatment to the patient terminal 20 at the set transmission timing. The question includes identification information of the electronic medical record as information for identifying the relevant treatment. When receiving, from the patient terminal 20, an answer to the question, i.e., information indicating post-treatment indicators for respective symptoms and input timings of the post-treatment indicators, the patient support unit 104 additionally stores the post-treatment indicators and the input timings included in the answer in the electronic medical record storage unit 303 in association with the identification information of the corresponding electronic medical record. Here, the patient support unit 104 may store the information in a storage unit different from the storage unit storing the electronic medical record. In any case, the information is stored in association with an information item such as an identifier of the electronic medical record or the treatment date to make it possible to identify the patient and the treatment for which the information is provided.

### 2.2.4. Electronic Medical Record Reference Process and Follow-up Information Reference Process

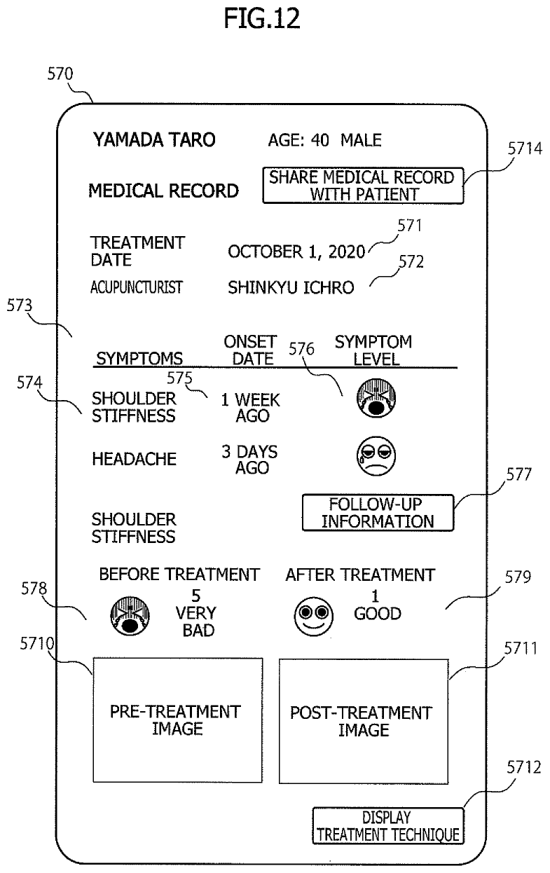

When the acupuncturist presses the details button 516 in the row of each electronic medical record on the patient details screen 510 of FIG. 5, i.e., when an electronic medical record including information indicating a treatment technique is specified, the electronic medical record output unit 105 displays an electronic medical record reference screen 570 illustrated as an example in FIG. 12 (S31 in FIG. 3). Also, the electronic medical record output unit 105 reads the electronic medical record from the electronic medical record storage unit 303 and displays the electronic medical record on the electronic medical record reference screen 570 (S32).

The electronic medical record reference screen 570 displays, in addition to patient information, a treatment date 571 and an attending acupuncturist 572. Also, the electronic medical record reference screen 570 displays symptoms 573 indicating the symptoms of the patient entered when the electronic medical record is generated, and a symptom list 574 that includes, for each symptom of the patient, an onset date 575 and a symptom level 576 indicating a pre-treatment indicator.

Also, the electronic medical record reference screen 570 displays a pre-treatment indicator 578 and a post-treatment indicator 579 for each symptom. Furthermore, the electronic medical record reference screen 570 displays a follow-up information button 577 for referring to the states of symptoms of the patient after treatment.

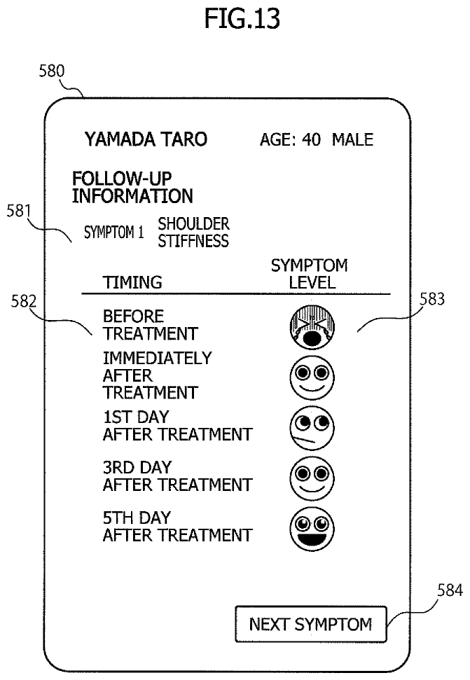

When the acupuncturist presses the follow-up information button 577, the electronic medical record output unit 105 displays a follow-up information screen 580 illustrated as an example in FIG. 13 (S33). Also, the electronic medical record output unit 105 reads and obtains pre-treatment indicators and post-treatment indicators from information included in or associated with the electronic medical record stored in the electronic medical record storage unit 303 together with information indicating the input timings of the indicators, and displays the obtained information in follow-up information 581 on the follow-up information screen 580 (S34). In the follow-up information 581, for each symptom, a pre-treatment indicator and/or post-treatment indicators and their input timings (each of which is indicated by, for example, the number of days counting from the treatment date or a day of the month) are arranged in chronological order in fields “date 582” and “symptom level 583”, respectively. Here, the electronic medical record output unit 105 does not necessarily display all of the obtained pre-treatment indicators and post-treatment indicators, and can display only some of the obtained pre-treatment indicators and post-treatment indicators entered on particular input timings.

The information displayed in the follow-up information 581 is based on information entered on the electronic medical record setting screen 540 (including information entered in a preliminary examination sheet and applied to an electronic medical record) and an answer from the patient terminal 20 to a question sent from the patient support unit 104 to the patient terminal 20. For example, the symptom levels displayed next to “Before treatment” and “Immediately after treatment” in the follow-up information 581 illustrated in FIG. 13 correspond to the pre-treatment indicator and the post-treatment indicator that are entered in the symptom level 547 before treatment and the symptom level 548 after treatment on the electronic medical record setting screen 540 of FIG. 9. Also, the symptom levels displayed next to “1st day after treatment”, “3rd day after treatment”, and “5th day after treatment” are post-treatment indicators obtained from the patient terminal 20. In the follow-up information 581, instead of displaying the pre-treatment indicator and the post-treatment indicators themselves as exemplified in FIG. 13, the pre-treatment indicator and the post-treatment indicators may be displayed in such a manner (for example, as a line graph) that the temporal transition of the pre-treatment indicator and the post-treatment indicators is visually recognizable. When multiple symptoms are set in an electronic medical record, the follow-up information screen 580 displays a “Next symptom” button 584 for displaying the follow-up information of the next symptom. When the “Next symptom” button 583 is pressed, the follow-up information of the next symptom is displayed (not shown). When, for example, a close button (not shown) is pressed, the follow-up information screen 580 is closed, and the electronic medical record reference screen 570 is displayed again (S35).

Referring back to FIG. 12, the electronic medical record reference screen 570 further displays a pre-treatment image field 5710 and a post-treatment image field 5711 for each symptom. In the example of the electronic medical record reference screen 570 of FIG. 12, although two symptoms including shoulder stiffness and headache are set, only image input fields for shoulder stiffness are displayed due to space limitations. The electronic medical record reference screen 570 also displays a treatment technique display button 5712 for displaying a treatment technique. When the treatment technique display button 5712 is pressed, the treatment technique set on the treatment technique setting screen 550 illustrated as an example in FIG. 10 are displayed (not shown).

The electronic medical record reference screen 570 further displays a “Share medical record with patient” button 5714. When the “Share medical record with patient” button 5714 is pressed, the electronic medical record setting unit 103 rewrites an access control flag of the electronic medical record stored in the electronic medical record storage unit 303 to give an access right to the patient terminal 20. This enables the patient terminal 20 to read and output the electronic medical record so that the patient can refer to the electronic medical record. In the example of FIG. 12, only one “Share medical record with patient” button 5714 is displayed for all items in the electronic medical record, and access control can be performed only in units of electronic medical records. However, it is also possible to control the access right for each item in the electronic medical record. For example, although not illustrated in the present embodiment, the electronic medical record generally includes a memo field for the acupuncturist. Access control may be performed such that the memo field is not shared with the patient. It is also possible to make a setting for sharing an electronic medical record at the same time as generating the electronic medical record.

### 2.2.5. Statistical Processing

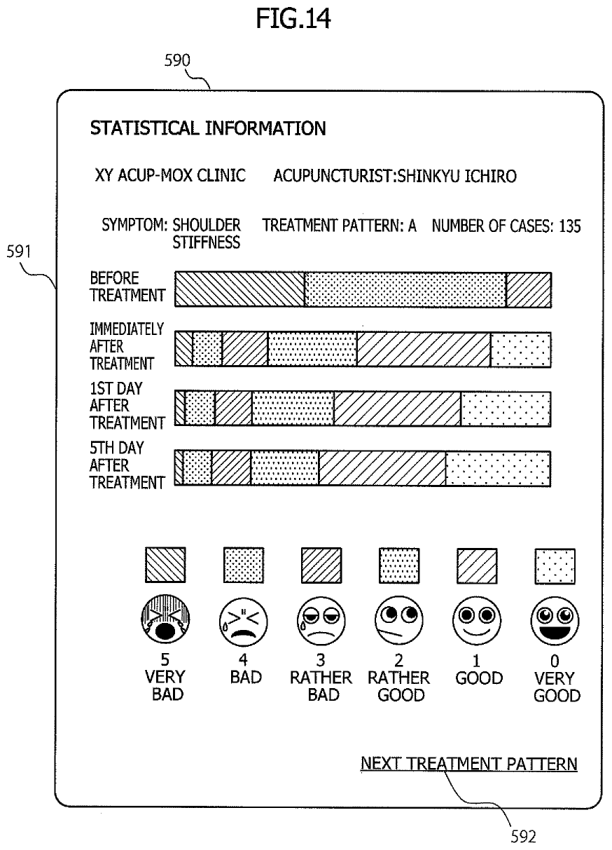

FIG. 14 illustrates an example of a statistical information screen 590 displayed by the statistical processing unit 106. The statistical processing unit 106 reads information stored in storage units such as electronic medical records stored in the electronic medical record storage unit 303, generates statistical information using various methods according to operations by the acupuncturist, and displays the statistical information on the statistical information screen 590 (S41 in FIG. 3). Also, the statistical processing unit 106 stores the generated statistical information in the statistical information storage unit 304 as necessary (S42).

For example, statistical information displayed on the statistical information screen 590 illustrated as an example in FIG. 14 is obtained based on collective data of electronic medical records accumulated in the electronic medical record storage unit 303 by grouping electronic medical records including the same symptom and the same treatment technique, performing statistical processing on pre-treatment indicators and post-treatment indicators for each group, and outputting the processing result in such a manner that the statistics on the pre-treatment indicators and the post-treatment indicators can be compared with each other for each treatment technique. More specifically, the statistical information is provided for a symptom “shoulder stiffness” treated by a treatment pattern A indicating a specific treatment technique and shows, using a band graph, the proportions of post-treatment indicators of patients for each of “before treatment”, “immediately after treatment”, “1st day after treatment”, and “5th day after treatment”. When a “To next treatment pattern” link at the bottom of the screen is tapped, a screen (not shown) similarly showing the proportions of post-treatment indicators with band graphs is displayed for a treatment pattern B indicating another treatment technique. By comparing these screens, the acupuncturist can determine how the pre-treatment indicators for the treatment pattern A and the post-treatment indicators for the treatment pattern B differ from each other. Also, with the example of FIG. 14, it is possible to determine the temporal change of the symptom of the patients in relation to the timing of treatment and to perform more detailed comparison. The processing result of the statistical processing is useful to determine which treatment technique is more appropriate, and enables the acupuncturist to select a treatment method with a higher treatment effect.

The statistical processing method used by the statistical processing unit 106 may be set in advance or may be constructed, as information is accumulated in storage units, by detecting correlations among various items based on accumulated big data. The statistical information illustrated as an example in FIG. 14 shows a temporal change in the proportions of the post-treatment indicators of a symptom of the patients based on all electronic medical records read from the electronic medical record storage unit 303. However, for example, when there are characteristic tendencies in the change of the symptom depending on attributes (for example, age, sex, occupation, and medical history) of the patients in the patient information included in the electronic medical records of the patients being treated, statistical processing may be performed for each of the attributes of the patients.

### 2.3. Details of Processes Performed by Patient Terminal

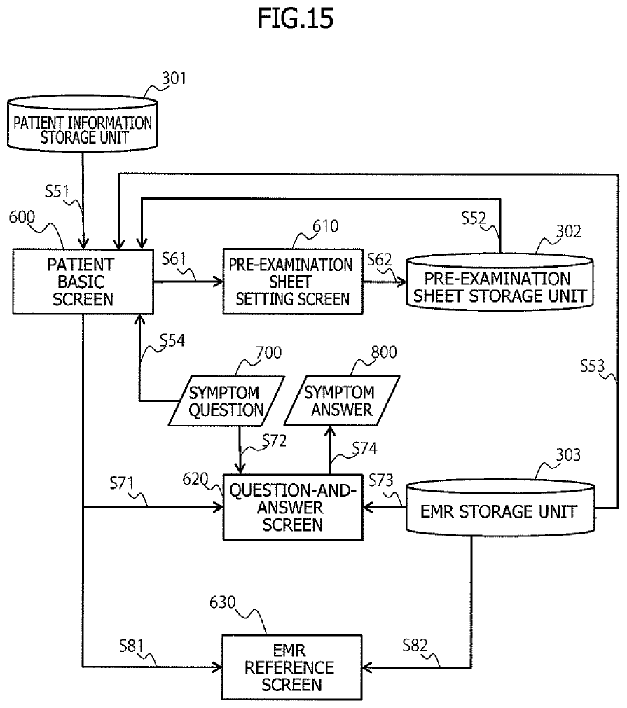

Next, processes performed by the preliminary examination sheet setting unit 201, the patient support answering unit 202, and the electronic medical record output unit 203 of the patient terminal 20 are described in detail with reference to examples of screens displayed in these processes. FIG. 15 is a drawing illustrating examples of processes performed by these units, along with screen transitions and access to storage units.

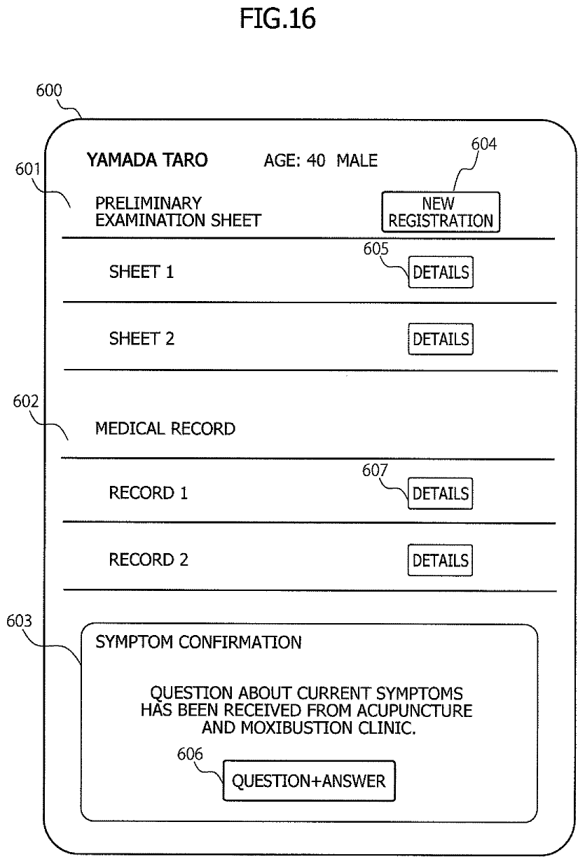

FIG. 16 illustrates an example of a patient basic screen 600 initially displayed on the patient terminal 20. A program (not shown) started on the patient terminal 20 reads patient information from the patient information storage unit 301 and displays the patient information on the patient basic screen 600 (S51 in FIG. 15). The preliminary examination sheet setting unit 201 reads information on preliminary examination sheets of the patient already stored in the preliminary examination sheet storage unit 302 and displays the preliminary examination sheets as a preliminary examination sheet list 601 (S52). Also, the electronic medical record output unit 203 reads information on electronic medical records of the patient already stored in the electronic medical record storage unit 303, and displays the information as an electronic medical record list 602 (S53). In this step, the electronic medical record output unit 203 refers to access control flags of electronic medical records and displays, in the list, only the information on electronic medical records for which an access right is given by the acupuncture and moxibustion clinic terminal 10.

Also, when a question (symptom question) 700 about symptoms has been received from the patient support unit 104 of the acupuncture and moxibustion clinic terminal 10 and has not been answered, the patient support answering unit 202 displays a question reception report 603 on the patient basic screen 600 (S54).

### 2.3.1. Preliminary Examination Sheet Setting Process

When a new registration button 604 for registering a new preliminary examination sheet is pressed by the acupuncturist on the patient basic screen 600, the preliminary examination sheet setting unit 201 displays a preliminary examination sheet setting screen 610 (S61 in FIG. 15). Because the preliminary examination sheet setting screen 610 is the same as the preliminary examination sheet setting screen 520 displayed by the acupuncture and moxibustion clinic terminal 10 as illustrated in FIG. 6, illustration of the preliminary examination sheet setting screen 610 is omitted. Also, because other screens and processes displayed and performed by the preliminary examination sheet setting unit 201 are basically the same as those displayed and performed by the preliminary examination sheet setting unit 102 of the acupuncture and moxibustion clinic terminal 10, their descriptions are omitted. Similarly to the preliminary examination sheet setting unit 102, the preliminary examination sheet setting unit 201 stores information entered on the preliminary examination sheet setting screen 610 in the preliminary examination sheet storage unit 302 in association with the patient information (S62). As a result, it becomes possible to refer to the preliminary examination sheet also from the acupuncture and moxibustion clinic terminal 10.

When the acupuncturist presses the details button 605 in the row of each preliminary examination sheet on the patient basic screen 600 in FIG. 16, the preliminary examination sheet setting unit 201 reads the preliminary examination sheet stored in the preliminary examination sheet storage unit 302 so that the preliminary examination sheet can be viewed on another screen. Although the preliminary examination sheet can be edited or deleted on the screen as necessary, descriptions of this function are omitted in this specification.

### 2.3.2. Patient Support Answering Process

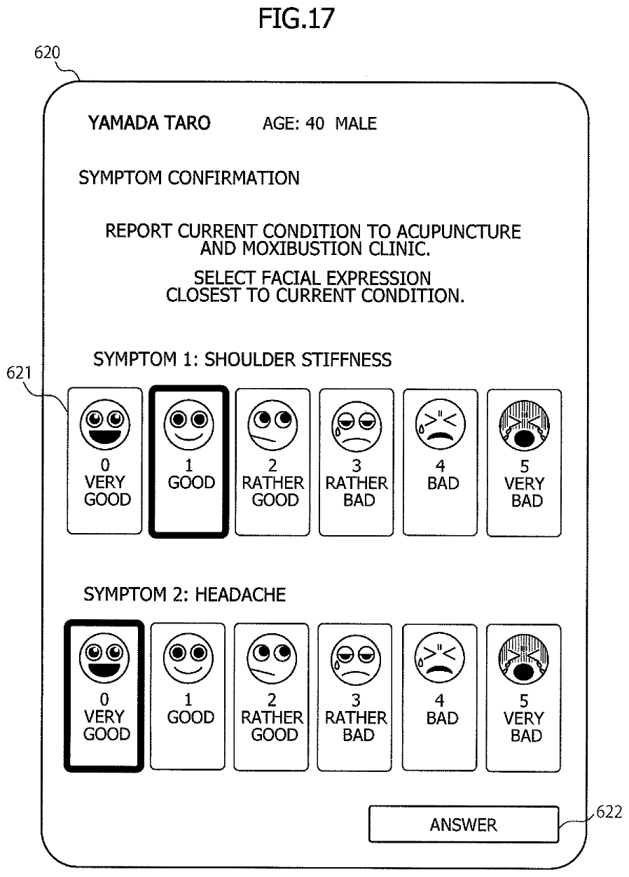

When a “Question+Answer” button 606 in the question reception report 603 is pressed by the patient on the patient basic screen 600, the patient support answering unit 202 displays a question-and-answer screen 620 illustrated as an example in FIG. 17 (S71 in FIG. 15). Based on the identification information of an electronic medical record included in a symptom question 700 sent by the patient support unit 104 of the acupuncture and moxibustion clinic terminal 10 (S72), the patient support answering unit 202 reads the corresponding electronic medical record stored in the electronic medical record storage unit 303 (S73). Then, for each symptom set in the electronic medical record, the patient support answering unit 202 displays, on the question-and-answer screen 620, indicator buttons 621 that display indicators representing symptom levels. The patient support answering unit 202 displays, on the question-and-answer screen 620, the indicator buttons 621 the number of which corresponds to the number of symptom levels set in the electronic medical record by the patient support unit 104 of the acupuncture and moxibustion clinic terminal 10. In the present embodiment, as illustrated as an example in FIG. 17, the number of symptom levels is set to six. Then, when the patient selects and presses one of the indicator buttons 621, the pressed indicator button 621 is highlighted (see, for example, the indicator button representing a symptom level “1: Good” of shoulder stiffness in FIG. 17).

When the patient presses the indicator buttons 621 for all symptoms on the question-and-answer screen 620 and presses an answer button 622, the patient support answering unit 202 generates a symptom answer 800 in which the post-treatment indicators represented by the pressed indicator buttons 621 and an input timing (which is indicated by, for example, the current date and time or the number of days that have passed from the treatment to the present) are associated with the corresponding symptoms, and sends the symptom answer 800 to the patient support unit 104 of the acupuncture and moxibustion clinic terminal 10 (S74).

### 2.3.3. Electronic Medical Record Reference Process and Follow-up Information Reference Process

When the patient presses a details button 607 in the row of each medical record on the patient basic screen 600, the electronic medical record output unit 203 displays an electronic medical record reference screen 630 (S81 in FIG. 15). Also, the electronic medical record output unit 203 reads the electronic medical record from the electronic medical record storage unit 303 and displays the electronic medical record on the electronic medical record reference screen 630 (S82). Because the electronic medical record reference screen 630 is the same as the electronic medical record reference screen 570 displayed by the acupuncture and moxibustion clinic terminal 10 as illustrated in FIG. 12, illustration of the electronic medical record reference screen 630 is omitted. Also, because other screens and processes displayed and performed by the electronic medical record output unit 203 are substantially the same as those displayed and performed in the electronic medical record reference process by the electronic medical record setting unit 103 of the acupuncture and moxibustion clinic terminal 10, their descriptions are omitted. Here, a screen similar to the follow-up information screen 580 illustrated in FIG. 13 may also be displayed by the electronic medical record output unit 203 of the patient terminal 20. Thus, the patient can refer to electronic medical records set by the acupuncture and moxibustion clinic terminal 10 and can also refer to follow-up information on symptoms of the patient that is set as a result of answering the question about the symptoms described above.

### 2.4. Details of Process Performed by Management Server

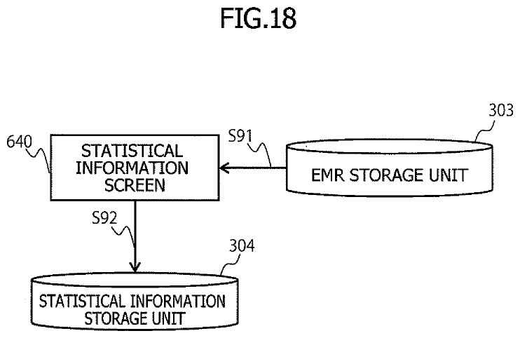

Next, a process performed by the statistical processing unit 401 of the management server 40 is described. FIG. 18 is a drawing illustrating an example of a process performed by the statistical processing unit 401 along with displayed screens and access to storage units. The statistical processing unit 401 reads information stored in storage units such as electronic medical records stored in the electronic medical record storage unit 303, generates statistical information using various methods according to operations by the acupuncturist, and displays the statistical information on a statistical information screen 640 (S91 in FIG. 18). Also, the statistical processing unit 401 stores the generated statistical information in the statistical information storage unit 304 as necessary (S92).

The process performed by the statistical processing unit 401 is substantially the same as the process performed by the statistical processing unit 106 of the acupuncture and moxibustion clinic terminal 10, and the statistical information screen 640 is substantially the same as the statistical information screen 590. Therefore, descriptions of the same parts of the process and illustration of the screen are omitted. The statistical processing unit 401 differs from the statistical processing unit 106 in that the statistical processing unit 401 can perform statistical processing for data of all acupuncture and moxibustion clinics. For example, the statistical information of FIG. 14 displayed on the statistical information screen 590 by the statistical processing unit 106 of the acupuncture and moxibustion clinic terminal 10 is based only on data of an acupuncture and moxibustion clinic in which the acupuncture and moxibustion clinic terminal 10 is used. In contrast, when, for example, treatment based on the same treatment template is performed using the same treatment technique at multiple acupuncture and moxibustion clinics, the statistical processing unit 401 can perform statistical processing based not only on data of pre-treatment indicators and post-treatment indicators accumulated by a specific acupuncture and moxibustion clinic, but also on the data accumulated by all of the acupuncture and moxibustion clinics performing the treatment. This in turn makes it possible to perform statistical processing based on a large amount of data and thereby makes it possible to obtain more reliable statistical information.

### 3. Effects and Variations of Present Embodiment

According to the present embodiment, symptoms of a patient before and after acupuncture and moxibustion treatment are represented and output using pre-treatment indicators and post-treatment indicators that are selected from indicators representing symptom levels on a scale. Therefore, even in acupuncture and moxibustion treatment in which it is difficult to determine a symptom level of a patient from the appearance, the acupuncturist can accurately determine a change in the symptom level of the patient before and after treatment by referring to the pre-treatment indicator and the post-treatment indicators. As a result, it becomes possible to clearly indicate the degree of improvement of a symptom achieved by the treatment, to improve the motivation of the acupuncturist, and to easily determine whether a treatment policy is appropriate. This in turn makes it possible to improve treatment accuracy.

Also, in the present embodiment, pre-treatment images and post-treatment images are set in an electronic medical record in association with pre-treatment indicators and post-treatment indicators and are displayed. This enables the acupuncturist to accurately determine a change in a symptom of the patient before and after treatment by referring to the electronic medical record.

Furthermore, in the present embodiment, pre-treatment indicators and post-treatment indicators entered on the patient terminal 20 can be obtained and referred to on the acupuncture and moxibustion clinic terminal 10. This facilitates smooth communication between the acupuncturist and the patient and enables the acupuncturist to easily determine the conditions of the patient.

In particular, enabling the acupuncture and moxibustion clinic terminal 10 to send a question to the patient terminal 20 after treatment and to obtain post-treatment indicators entered on the patient terminal 20 makes it possible to easily determine a change in a symptom of the patient after treatment and enables the acupuncturist to follow up the conditions of the patient. Here, the patient can also enter symptom levels by selecting and entering post-treatment indicators. This reduces the burden of answering a question and makes it possible to accurately express the symptom levels. Also, because obtained post-treatment indicators are displayed in chronological order together with their input timings, the acupuncturist can visually and accurately determine how the symptom level of the patient has changed over time. Here, instead of the function for outputting pre-treatment indicators and post-treatment indicators described above, a function for obtaining post-treatment indicators from the patient terminal 20 may be provided as described above. This also makes it possible to achieve a purpose of enabling the acupuncturist to accurately determine the symptom level of a patient and improving the treatment accuracy in acupuncture and moxibustion treatment.

Furthermore, because the acupuncturist can freely set a follow-up period, i.e., a transmission period and a transmission interval for sending a question, for each case of treatment when generating an electronic medical record, the number of times symptom follow-up information is obtained can be adjusted according to the type of symptom and the level of the symptom. For example, for a patient with severe symptoms, a long transmission period and a short transmission interval may be set. The timing to send a question may not necessarily be a regular transmission interval, and any intervals such as a first day, a third day, a first week, and a second week after treatment may be set.

Also, the number of indicators in a scale from which the patient selects a post-treatment indicator to answer a question can be freely set by the acupuncturist when generating an electronic medical record. For example, this makes it possible to set a scale with a small number of indicators (for example, including only two levels: “Good” and “Bad”) for a patient for which it seems difficult to select an appropriate indicator from a large number of indicators.

Also, in the present embodiment, a preliminary examination sheet can be set not only from the acupuncture and moxibustion clinic terminal 10, but also from the patient terminal 20. This enables a patient to enter a preliminary examination sheet in advance at any timing that is convenient for the patient. Also, when entering a symptom in the preliminary examination sheet, the patient can enter a level of the symptom of the patient using a pre-treatment indicator. This enables the patient to easily and accurately tell the level of the symptom before treatment to the acupuncturist.

Furthermore, in the present embodiment, access rights are controlled to permit the patient terminal 20 to read all or some of items included in an electronic medical record set by the acupuncture and moxibustion clinic terminal 10. This makes it possible to allow the patient to refer to an electronic medical record created by the acupuncturist, and thereby makes it possible to increase the transparency of treatment, to reduce anxiety and ambiguity about acupuncture and moxibustion treatment, and to make acupuncture and moxibustion treatment more approachable for the patient.

Also, in the present embodiment, statistical processing is performed based on information indicating treatment techniques, pre-treatment indicators, and post-treatment indicators that are accumulated in storage units. This makes it possible to understand the overall tendency about what types of changes in symptoms are observed by performing what types of treatment techniques. As described above, the processing results of such statistical processing are useful to determine which treatment techniques are more appropriate, and enable the acupuncturist to select a treatment method with a higher treatment effect. The processing results of statistical processing can also be used to check the proficiency level of the acupuncturist. Also, it is possible to provide, for example, a function to suggest an appropriate treatment method as necessary or recommend participating in a seminar for a skill with insufficient proficiency. Furthermore, it is possible to use the processing results for the recruitment of acupuncturists. It is also possible to perform such statistical processing and output the processing results in place of a process for outputting pre-treatment indicators and post-treatment indicators.

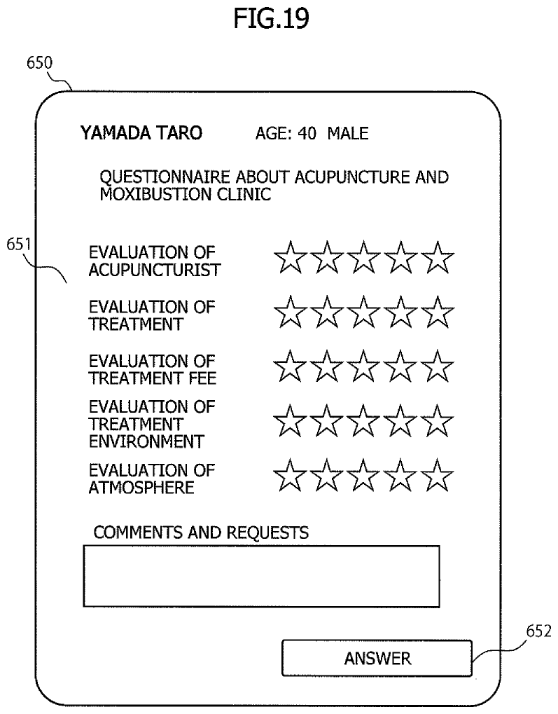

As a variation of the acupuncture and moxibustion treatment support system, the patient terminal 20 may be configured to also evaluate the acupuncture and moxibustion clinic after treatment and send the evaluation result to the acupuncture and moxibustion clinic terminal 10. FIG. 19 is an example of an acupuncture and moxibustion clinic evaluation screen 650 that can be displayed on the patient terminal 20 by the patient support answering unit 202. The acupuncture and moxibustion clinic evaluation screen 650 displays various evaluation items 651 as a questionnaire about an acupuncture and moxibustion clinic. The patient can enter an evaluation result expressed by the number of stars for each of the evaluation items 651. Then, when an answer button 652 is pressed by the patient who has completed entering evaluation results, the patient support answering unit 202 sends evaluation information including the entered evaluation results to the acupuncture and moxibustion clinic terminal 10. At the acupuncture and moxibustion clinic terminal 10 receiving the evaluation information, for example, the statistical processing unit 106 may store the evaluation information received and obtained from the patient terminal 20 in a storage unit of the data server 30, perform statistical processing on the evaluation information as necessary, and output the evaluation information on the statistical information screen 590. This enables the acupuncture and moxibustion clinic to know how the acupuncture and moxibustion clinic is evaluated by patients and to provide acupuncture and moxibustion treatment and services with higher quality by taking appropriate measures. Also, similar statistical processing can be performed by the statistical processing unit 401 of the management server 40.

In the present embodiment, information indicating treatment techniques, pre-treatment indicators, and post-treatment indicators are included in an electronic medical record. However, the present invention is not limited to this embodiment, and the information indicating treatment techniques, pre-treatment indicators, and post-treatment indicators may be set in a storage unit as treatment information different from the electronic medical record. Also, information such as pre-treatment indicators and post-treatment indicators may be displayed together with information indicating treatment techniques on a screen separate from the screen for displaying the electronic medical record.

### [Hardware Configuration of Information Processing Apparatus]

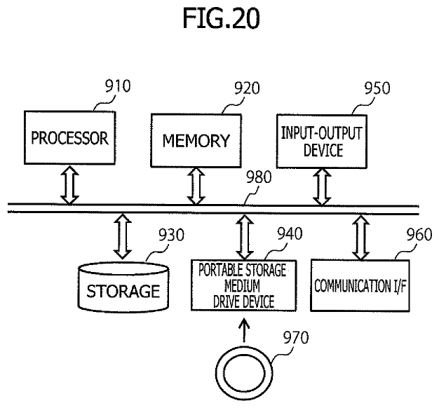

FIG. 20 illustrates an example of a hardware configuration of an information processing apparatus implementing each of the acupuncture and moxibustion clinic terminal 10, the patient terminal 20, and the data server 30 that constitute the acupuncture and moxibustion treatment support system of the present embodiment. The information processing apparatus includes a processor 910, a memory 920, a storage 930, a portable storage medium drive device 940, an input-output device 950, and a communication interface 960.

The processor 910 includes a control unit, an arithmetic unit, and an instruction decoder. In the processor 910, an execution unit causes the arithmetic unit to perform arithmetic and logical operations according to instructions of a program decoded by the instruction decoder in response to a control signal output from the control unit. The processor 910 includes a control register for storing various types of information used for control, and a cache capable of temporarily storing information that is contained in, for example, the memory 920 and has already been accessed. The processor 910 may have a configuration that includes multiple central processing unit (CPU) cores.

The memory 920 is, for example, a storage device such as a random-access memory (RAM) and is a main memory into which programs to be executed by the processor 910 are loaded and that stores data used in processes performed by the processor 910. The storage 930 is a storage device such as a hard disk drive (HDD) or a flash memory and stores programs and various types of data. The portable storage medium drive device 940 reads data and programs stored in a portable storage medium 970. The portable storage medium 970 is, for example, a magnetic disk, an optical disk, a magneto-optical disk, or a flash memory. The processor 910 executes programs stored in the storage 930 and the portable storage medium 970 in corporation with the memory 920 and the storage 930. Here, programs to be executed and data to be accessed by the processor 910 may be stored in another device that can communicate with the information processing apparatus.

The input-output device 950 functions as an input unit and an output unit and is implemented by, for example, a touch panel, a keyboard, and/or a display. The input-output device 950 receives operation commands corresponding to operations performed by the user, and outputs results of processes performed by the information processing apparatus. The communication interface 960 is a communication module such as the communication unit 122 of the acupuncture and moxibustion clinic terminal 10 or the communication unit 212 of the patient terminal 20 described above, and enables data communications with an external device. The components of the information processing apparatus described above are connected to each other via a bus 980.

### [Other Matters]

The functional configuration and the physical configuration of the information processing apparatus described in this specification are not limited to those described above. For example, functions and physical resources described above may be integrated or, conversely, may be further distributed for implementation.

### REFERENCE SYMBOL LIST

10 . . . acupuncture and moxibustion clinic terminal, 101 . . . patient information setting unit, 102 . . . preliminary examination sheet setting unit, 103 . . . electronic medical record setting unit, 104 . . . patient support unit, 105 . . . electronic medical record output unit, 106 . . . statistical processing unit, 20 . . . patient terminal, 201 . . . preliminary examination sheet setting unit, 202 . . . patient support answering unit, 203 . . . electronic medical record output unit, 30 . . . data server, 301 . . . patient information storage unit, 302 . . . preliminary examination sheet storage unit, 303 . . . electronic medical record storage unit, 304 . . . statistical information storage unit, 40 . . . management server, 401 . . . statistical processing unit

1. An acupuncture and moxibustion treatment support apparatus comprising:
a treatment information setting unit that
obtains a pre-treatment indicator corresponding to a symptom level of a patient before treatment and a post-treatment indicator corresponding to a symptom level of the patient after treatment that are entered based on selection by the patient from indicators representing symptom levels of a symptom treated by acupuncture and moxibustion treatment, the patient receiving the acupuncture and moxibustion treatment, and
stores the pre-treatment indicator and the post-treatment indicator in a storage unit in association with information indicating a treatment technique used in the acupuncture and moxibustion treatment; and
a treatment information output unit that when the information indicating the treatment technique is specified, reads the information indicating the treatment technique, the pre-treatment indicator, and the post-treatment indicator from the storage unit and outputs the information indicating the treatment technique, the pre-treatment indicator, and the post-treatment indicator.

2. The acupuncture and moxibustion treatment support apparatus as claimed in claim 1, wherein
the treatment information setting unit also stores a pre-treatment image indicating the symptom of the patient before treatment and a post-treatment image indicating the symptom of the patient after treatment in the storage unit; and
the treatment information output unit reads the pre-treatment image and the post-treatment image from the storage unit and outputs the pre-treatment image and the post-treatment image in association with the pre-treatment indicator and the post-treatment indicator.

3. The acupuncture and moxibustion treatment support apparatus as claimed in claim 1, wherein the treatment information setting unit obtains, from the patient terminal used by the patient, at least one of the pre-treatment indicator and the post-treatment indicator entered on the patient terminal.

4. The acupuncture and moxibustion treatment support apparatus as claimed in claim 3, further comprising:
a patient support unit that after the acupuncture and moxibustion treatment is performed, obtains, from the patient terminal, the post-treatment indicator entered on the patient terminal together with information indicating an input timing of the post-treatment indicator and in association with the information indicating the treatment technique.

5. The acupuncture and moxibustion treatment support apparatus as claimed in claim 4, wherein after the acupuncture and moxibustion treatment is performed, the patient support unit sends a question about the symptom of the patient after treatment to the patient terminal and obtains, from the patient terminal, the post-treatment indicator entered on the patient terminal in response to the question together with the information indicating the input timing.

6. The acupuncture and moxibustion treatment support apparatus as claimed in claim 5, wherein the treatment information output unit outputs the pre-treatment indicator and all or some of post-treatment indicators obtained from the patient terminal, in chronological order, together with corresponding input timings.

7. The acupuncture and moxibustion treatment support apparatus as claimed in claim 5, wherein the patient support unit sends the question at a timing based on a preset transmission period and a preset transmission interval.

8. The acupuncture and moxibustion treatment support apparatus as claimed in claim 5, wherein the patient support unit sends the question at a timing that is entered and set for each case of the acupuncture and moxibustion treatment.

9. The acupuncture and moxibustion treatment support apparatus as claimed in claim 3, wherein the treatment information setting unit obtains the pre-treatment indicator included in a preliminary examination sheet obtained from the patient terminal before the acupuncture and moxibustion treatment.

10. The acupuncture and moxibustion treatment support apparatus as claimed in claim 3, wherein the treatment information setting unit stores an electronic medical record including the information indicating the treatment technique, the pre-treatment indicator, and the post-treatment indicator in the storage unit and controls an access right to permit the patient terminal to read all or some of items included in the electronic medical record.

11. The acupuncture and moxibustion treatment support apparatus as claimed in claim 1, further comprising:
a statistical processing unit that performs statistical processing based on sets of information indicating treatment techniques, pre-treatment indicators, and post-treatment indicators that are accumulated in the storage unit for multiple cases of the acupuncture and moxibustion treatment, and outputs a processing result of the statistical processing.

12. The acupuncture and moxibustion treatment support apparatus as claimed in claim 11, wherein the statistical processing unit groups sets of information including the same symptom and the same treatment technique, performs statistical processing on the pre-treatment indicators and the post-treatment indicators for each group, and outputs processing results in such a manner that results of the statistical processing on the pre-treatment indicators and the post-treatment indicators can be compared with each other for each treatment technique.

13. The acupuncture and moxibustion treatment support apparatus as claimed in claim 11, wherein the statistical processing unit also obtains, from the patient terminal, information indicating evaluation results of an acupuncture and moxibustion clinic at which the acupuncture and moxibustion treatment has been performed, performs statistical processing on the evaluation results, and outputs a processing result of the statistical processing.

14. An acupuncture and moxibustion treatment support method performed by an information processing apparatus, the method comprising:
obtaining a pre-treatment indicator corresponding to a symptom level of a patient before treatment and a post-treatment indicator corresponding to a symptom level of the patient after treatment that are entered based on selection by the patient from indicators representing symptom levels of a symptom treated by acupuncture and moxibustion treatment, the patient receiving the acupuncture and moxibustion treatment;
storing the pre-treatment indicator and the post-treatment indicator in a storage unit in association with information indicating a treatment technique used in the acupuncture and moxibustion treatment;
reading the information indicating the treatment technique, the pre-treatment indicator, and the post-treatment indicator from the storage unit when the information indicating the treatment technique is specified; and
outputting the information indicating the treatment technique, the pre-treatment indicator, and the post-treatment indicator.

15. A computer-readable, non-transitory medium storing an acupuncture and moxibustion treatment support program for causing a computer to execute a process including:
obtaining a pre-treatment indicator corresponding to a symptom level of a patient before treatment and a post-treatment indicator corresponding to a symptom level of the patient after treatment that are entered based on selection by the patient from indicators representing symptom levels of a symptom treated by acupuncture and moxibustion treatment, the patient receiving the acupuncture and moxibustion treatment;
storing the pre-treatment indicator and the post-treatment indicator in a storage unit in association with information indicating a treatment technique used in the acupuncture and moxibustion treatment;
reading the information indicating the treatment technique, the pre-treatment indicator, and the post-treatment indicator from the storage unit when the information indicating the treatment technique is specified; and
outputting the information indicating the treatment technique, the pre-treatment indicator, and the post-treatment indicator.

16. An acupuncture and moxibustion treatment support system comprising:
a patient terminal used by a patient receiving acupuncture and moxibustion treatment; and
an acupuncture and moxibustion clinic terminal used at an acupuncture and moxibustion clinic providing the acupuncture and moxibustion treatment, wherein
after the acupuncture and moxibustion treatment is performed, the patient terminal provides, to the acupuncture and moxibustion clinic terminal, a post-treatment indicator that corresponds to a symptom level of the patient after treatment and is entered based on selection by the patient from indicators representing symptom levels of a symptom; and
the acupuncture and moxibustion clinic terminal outputs the post-treatment indicator obtained from the patient terminal in association with information indicating a treatment technique used in the acupuncture and moxibustion treatment.

17. The acupuncture and moxibustion treatment support system as claimed in claim 16, wherein
after the acupuncture and moxibustion treatment is performed, the acupuncture and moxibustion clinic terminal sends a question about the symptom of the patient after treatment to the patient terminal; and
upon receiving the question from the acupuncture and moxibustion clinic terminal, the patient terminal provides the post-treatment indicator, which is entered based on the selection by the patient, to the acupuncture and moxibustion clinic terminal.

18. An acupuncture and moxibustion treatment support system comprising:
one or more patient terminals used by patients receiving acupuncture and moxibustion treatment;
one or more acupuncture and moxibustion clinic terminals used at one or more acupuncture and moxibustion clinics providing the acupuncture and moxibustion treatment;
a data server including a storage unit storing information written into the storage unit by the acupuncture and moxibustion clinic terminals and the patient terminals; and
a management server capable of accessing the storage unit, wherein
the acupuncture and moxibustion clinic terminals
obtain pre-treatment indicators corresponding to symptom levels of the patients before treatment and post-treatment indicators corresponding to symptom levels of the patients after treatment that are entered, using the acupuncture and moxibustion clinic terminals or the patient terminals, based on selection by the patients from indicators representing symptom levels of symptoms treated by the acupuncture and moxibustion treatment, and
store the pre-treatment indicators and the post-treatment indicators in the storage unit in association with information indicating treatment techniques used in the acupuncture and moxibustion treatment; and
the management server performs statistical processing based on the information indicating the treatment techniques, the pre-treatment indicators, and the post-treatment indicators that are accumulated in the storage unit for multiple cases of the acupuncture and moxibustion treatment, and outputs a processing result of the statistical processing.
